SEQUENCE LISTING (31 sequences)

SEQ ID NO: 1 - DNA, 26 bp, Artificial Sequence
  FP_ZIKA_San0-F
aartacacataccaraacaaagtggt

SEQ ID NO: 2 - DNA, 23 bp, Artificial Sequence
  FP_ZIKV_San0.alt-F
tacacataccaraacaaagtggt

SEQ ID NO: 3 - DNA, 23 bp, Artificial Sequence
  RP_ZIKA_San0-R
acttgtccrctcccyctytggtc

SEQ ID NO: 4 - DNA, 22 bp, Artificial Sequence
  FP_ZIKA_San-3F
tggtgtggaayagrgtgtggat

SEQ ID NO: 5 - DNA, 22 bp, Artificial Sequence
  RP_ZIKA_San-3R
gtayttytcttcatcacctatg

SEQ ID NO: 6 - DNA, 20 bp, Artificial Sequence
  FP_ZIKA_San-1F
gttgtggatggaatagtggt

SEQ ID NO: 7 - DNA, 19 bp, Artificial Sequence
  RP_ZIKA_San-1R
agtarcacytgtcccatct

SEQ ID NO: 8 - DNA, 21 bp, Artificial Sequence
  probe
  misc_feature (16)..(16): preferably LNA
  misc_feature (17)..(17): preferably LNA
  misc_feature (18)..(18): preferably LNA
  misc_feature (19)..(19): preferably LNA
aaggtyctyagaccagctgaa

SEQ ID NO: 9 - DNA, 17 bp, Artificial Sequence
  probe
  misc_feature (5)..(5): preferably LNA
  misc_feature (8)..(8): preferably LNA
  misc_feature (14)..(14): preferably LNA
cgcaccachtgggctga

SEQ ID NO: 10 - DNA, 23 bp, Artificial Sequence
  probe
actgacattgacacaatgacaat

SEQ ID NO: 11 - DNA, 20 bp, Artificial Sequence
  probe
  misc_feature (5)..(5): preferably LNA
  misc_feature (6)..(6): preferably LNA
  misc_feature (10)..(10): preferably LNA
aaatggacagacattcccta

SEQ ID NO: 12 - DNA, 21 bp, Artificial Sequence
  RP_ZIKV 9352-R
tccrctcccyctytggtcttg

SEQ ID NO: 13 - DNA, 16 bp, Artificial Sequence
  Probe ZIKV 9304-P
  misc_feature (10)..(10): preferably LNA
  misc_feature (11)..(11): preferably LNA
  misc_feature (12)..(12): preferably LNA
  misc_feature (13)..(13): preferably LNA
ctyagaccagctgaar

SEQ ID NO: 14 - DNA, 23 bp, Artificial Sequence
  RP_ZIKA_San0-AltB
cttgaratratgtccatractgt

SEQ ID NO: 15 - DNA, 20 bp, Artificial Sequence
  FP_ZIKA_San-2F
gayggkagrtccattgtggt

SEQ ID NO: 16 - DNA, 21 bp, Artificial Sequence
  RP_ZIKA_San-2R
agtctcmcgratgctccatcc

SEQ ID NO: 17 - DNA, 26 bp, Artificial Sequence
  Probe_ZIKA_San-2P
cgccaccaagatgaaytgattggccg

SEQ ID NO: 18 - DNA, 20 bp, Artificial Sequence
  FP_ZIKA_San-3Fb
aartggacagahatycccta

SEQ ID NO: 19 - DNA, 23 bp, Artificial Sequence
  Probe_ZIKA_San-3P
  misc_feature (15)..(15): n is a, c, g, or t
atagggcacagrccncgcacyac

SEQ ID NO: 20 - DNA, 17 bp, Artificial Sequence
  FP_ZIKA_San-4F
ttagctggkccactcag

SEQ ID NO: 21 - DNA, 20 bp, Artificial Sequence
  RP_ZIKA_San-4R
gttccrcatgtytcctccac

SEQ ID NO: 22 - DNA, 29 bp, Artificial Sequence
  Probe_ZIKA_San-4P
agtgaagagctygaaatycggtttgagga

SEQ ID NO: 23 - DNA, 20 bp, Artificial Sequence
  FP_ZIKA_San-5F
gtggaggaracatgyggaac

SEQ ID NO: 24 - DNA, 20 bp, Artificial Sequence
  RP_ZIKA_San-5R
cagcaccattcctcdatsac

SEQ ID NO: 25 - DNA, 25 bp, Artificial Sequence
  Probe_ZIKA_San-5P
ggaagggtsathgaggaatggtgct

SEQ ID NO: 26 - DNA, 6 bp, Artificial Sequence
  oligonucleotice
gtaytr

SEQ ID NO: 27 - DNA, 6 bp, Artificial Sequence
  oligonucleotice
gtaata

SEQ ID NO: 28 - DNA, 6 bp, Artificial Sequence
  oligonucleotice
gtaatg

SEQ ID NO: 29 - DNA, 6 bp, Artificial Sequence
  oligonucleotice
gtatta

SEQ ID NO: 30 - DNA, 6 bp, Artificial Sequence
  oligonucleotice
gtattg

SEQ ID NO: 31 - DNA, 17 bp, Artificial Sequence
  Probe_ZIKA-Reverse
tcagcccadgtggtgcg

(19) United States
(12) Patent Application Publication
(10) Pub. No.: US 2019/0264294 A1
(43) Pub. Date: Aug. 29, 2019

(54) DETECTION OF ZIKA VIRUS NUCLEIC ACID

(71) Applicant: Stichting Sanquin Bloedvoorziening, Amsterdam (NL)

(72) Inventors: Marcus Henricus Gerardus Maria Koppelman, Amstelveen (NL); Mohamed Boujnan, Haarlem (NL)

(21) Appl. No.: 16/088,019

(22) PCT Filed: Mar. 24, 2017
(86) PCT No.: PCT/NL2017/050185

§ 371 Date: Sep. 24, 2018

(30) Foreign Application Priority Data
Mar. 24, 2016 (EP) 16162434.1

(51) Int. Cl. C12Q 1/70

(52) U.S. Cl. CPC C12Q 1/701

(57) ABSTRACT

The disclosure describes methods, kits, apparatus and oligonucleotides for the detection of Zika virus. In one aspect, the disclosure describes a method of determining whether a sample comprises ZIKA virus (ZIKV) nucleic acid comprising performing an amplification reaction with the sample in the presence of a second oligonucleotide set, a first oligonucleotide set, a third oligonucleotide set or a combination of two or more of the sets, wherein: the first oligonucleotide set has: an oligonucleotide ON A with a consecutive stretch of at least 18 nucleotides of the sequence AARTACACATACCARAACAAAGTGGT (FP_ZIKA_San0-F) (SEQ ID NO:1) or a consecutive stretch of at least 18 nucleotides of the sequence TACACATACCARAACAAAGTGGT (FP_ZIKV_San0.alt-F) (SEQ ID NO:2); and an oligonucleotide ON B with a consecutive stretch of at least 18 nucleotides of the sequence ACTTGTCCRCTCCCYCTYTGGTC (RP_ZIKA_San0-R) (SEQ ID NO:3); the second oligonucleotide set has: an oligonucleotide ON C with a consecutive stretch of at least 18 nucleotides of the sequence TGGTGTGGAAYAGRGTGTGGAT (FP_ZIKA_San-3F) (SEQ ID NO:4); and an oligonucleotide ON D with a consecutive stretch of at least 18 nucleotides of the sequence GTAYTTYTCTTCATCACCTATG (RP_ZIKA_San-3R) (SEQ ID NO:5); and the third oligonucleotide set has: an oligonucleotide ON E with a consecutive stretch of at least 18 nucleotides of the sequence GTTGTGGATGGAATAGTGGT (FP_ZIKA_San-1F) (SEQ ID NO:6); and an oligonucleotide ON F with a consecutive stretch of at least 18 nucleotides of the sequence AGTARCACYTGTCCCATCT (RP_ZIKA_San-1R) (SEQ ID NO:7); the method further comprising determining whether the sample comprises an amplification product of the amplification reaction.

### CROSS-REFERENCE TO RELATED APPLICATIONS

This application is a national phase entry under 35 U.S.C. § 371 of International Patent Application PCT/NL2017/050185, filed Mar. 24, 2017, designating the United States of America and published in English as International Patent Publication WO 2017/164741 A2 on Sep. 28, 2017, which claims the benefit under Article 8 of the Patent Cooperation Treaty to European Patent Application Serial No. 16162434.1, filed Mar. 24, 2016.

### TECHNICAL FIELD

The disclosure relates to methods, kits, apparatuses and oligonucleotides for the detection of Zika virus.

### STATEMENT ACCORDING TO 37 C.F.R. § 1.821(c) or (e)—SEQUENCE LISTING SUBMITTED AS ASCII TEXT FILE

Pursuant to 37 C.F.R. § 1.821(c) or (e), a file containing an ASCII text version of the Sequence Listing has been submitted concomitant with this application, the contents of which are hereby incorporated by reference.

### BACKGROUND

Zika virus (ZIKV) is an arbovirus that is transmitted by several species of Aedes mosquitoes, but especially by Aedes albopictus (Asian tiger mosquito) and Aedes aegypti (yellow fever mosquito). The clinical symptoms of infection with ZIKV range from none (80%) to mild symptoms (e.g., headache, fever, myalgia, and/or arthralgia) and resemble the clinical symptoms after a Dengue virus and Chikungunya virus infection. A link has been established with neurologic conditions in infected adults, including Guillain-Barré syndrome. ZIKA virus infections are also associated with microcephaly in newborns through mother to child transmission. ZIKV is sexually transmitted and transmitted by blood.

ZIKV virus is known in Africa and Asia in the areas around the equator since the fifties. In 2014, ZIKV spread to areas in the Pacific (French Polynesia) and then in 2015 to South America, the Caribbean, Mexico and Central America. In these areas, an epidemic of ZIKV is now in progress. In January 2016, the World Health Organization (WHO), declared the ZIKV epidemic an international emergency.

During the first week after onset of symptoms, Zika virus nucleic acids can often be detected in the serum or plasma. Virus-specific IgM and neutralizing antibodies typically develop toward the end of the first week of illness; cross-reaction with related flaviviruses (e.g., dengue and yellow fever viruses) is common and may be difficult to discern. Plaque-reduction neutralization testing can be performed to measure virus-specific neutralizing antibodies and discriminate between cross-reacting antibodies in primary Flavivirus infections. Although these methods allow the definitive determination of ZIKV infection, the present methods are cumbersome and most importantly not suited for mass screening of, for instance, blood donor samples.

### BRIEF SUMMARY

Described is a nucleic acid amplification based detection method. The method is fast, robust and able to detect a wide variety of ZIKV strains. The method does not detect the evolutionary related dengue and yellow fever flaviviruses.

The disclosure provides a method of determining whether a sample comprises ZIKA virus (ZIKV) nucleic acid the method comprising performing an amplification reaction with the sample in the presence of a first oligonucleotide set, a second oligonucleotide set, a third oligonucleotide set or a combination of two or more of the sets, wherein:
the first oligonucleotide set has:
   an oligonucleotide ON A with a consecutive stretch of at least 18 nucleotides of the sequence AARTACACATACCARAACAAAGTGGT (FP_ZIKA_San0-F) (SEQ ID NO:1) or a consecutive stretch of at least 18 nucleotides of the sequence TACACATACCARAACAAAGTGGT (FP_ZIKV_San0.alt-F) (SEQ ID NO:2); and
   an oligonucleotide ON B with a consecutive stretch of at least 18 nucleotides of the sequence ACTTGTCCRCTCCCYCTYTGGTC (RP_ZIKA_San0-R) (SEQ ID NO:3);
the second oligonucleotide set has:
   an oligonucleotide ON C with a consecutive stretch of at least 18 nucleotides of the sequence TGGTGTGGAAYAGRGTGTGGAT (FP_ZIKA_San-3F) (SEQ ID NO:4); and
   an oligonucleotide ON D with a consecutive stretch of at least 18 nucleotides of the sequence GTAYTTYTCTTCATCACCTATG (RP_ZIKA_San-3R) (SEQ ID NO:5); and
the third oligonucleotide set has:
   an oligonucleotide ON E with a consecutive stretch of at least 18 nucleotides of the sequence GTTGTGGATGGAATAGTGGT (FP_ZIKA_San-1F) (SEQ ID NO:6);
   an oligonucleotide ON F with a consecutive stretch of at least 18 nucleotides of the sequence AGTARCACYTGTCCCATCT (RP_ZIKA_San-1R) (SEQ ID NO:7);
in which R=A+G; Y=C+T,
the method further comprising determining whether the sample comprises an amplification product of the amplification reaction.

The disclosure also provides a kit comprising a first oligonucleotide set, a second oligonucleotide set, a third oligonucleotide set or a combination of two or more of the sets, wherein:
the first oligonucleotide set has:
   an oligonucleotide ON A with a consecutive stretch of at least 18 nucleotides of the sequence AARTACACATACCARAACAAAGTGGT (FP_ZIKA_San0-F) (SEQ ID NO:1) or a consecutive stretch of at least 18 nucleotides of the sequence TACACATACCARAACAAAGTGGT (FP_ZIKV_San0.alt-F) (SEQ ID NO:2); and
   an oligonucleotide ON B with a consecutive stretch of at least 18 nucleotides of the sequence ACTTGTCCRCTCCCYCTYTGGTC (RP_ZIKA_San0-R) (SEQ ID NO:3);
the second oligonucleotide set has:
   an oligonucleotide ON C with a consecutive stretch of at least 18 nucleotides of the sequence TGGTGTGGAAYAGRGTGTGGAT (FP_ZIKA_San-3F) (SEQ ID NO:4); and
an oligonucleotide ON D with a consecutive stretch of at least 18 nucleotides of the sequence GTAYTTYTCTTCATCACCTATG (RP_ZIKA_San-3R) (SEQ ID NO:5); and

the third oligonucleotide set has:
an oligonucleotide ON E with a consecutive stretch of at least 18 nucleotides of the sequence GTTGTGGATGGAATAGTGGT (FP_ZIKA_San-1F) (SEQ ID NO:6);
an oligonucleotide ON F with a consecutive stretch of at least 18 nucleotides of the sequence AGTARCACYTGTCCCATCT (RP_ZIKA_San-1R) (SEQ ID NO:7),

in which R=A+G; Y=C+T.

As used herein, the term “sample” as used in its broadest sense to refer to any biological sample from any human or veterinary subject that may be tested for the presence or absence of Zika virus nucleic acid. The samples may include, without limitation, tissues obtained from any organ, such as for example, lung tissue; and fluids obtained from any organ such as for example, blood, plasma, serum, urine, lymphatic fluid, synovial fluid, cerebrospinal fluid, amniotic fluid, amniotic cord blood, tears, saliva, and nasopharyngeal washes. The sample is preferably a biological sample of an individual. Preferably the sample is a body fluid sample such as whole blood, serum, plasma, urine, semen or sputum sample. The sample can also be a stool sample or a cell or cell culture sample.

The sample is preferably from a human or animal. The animal is preferably a mammal.

Zika virus has been known for quite some time. Various strains are presently recognized. The strain that is marked the Asian strain is thought to be responsible for the recent epidemic outbreak in the Americas (Enffisi et al., The Lancet, Volume 387, No. 10015, pp. 227-228, 16 Jan. 2016). Zika virus is an RNA virus of the Flavivirus genus. As such, it rapidly incorporates mutations resulting in deviation from the prototype virus at one or more positions. Some regions of the genome are more resistant to such mutations, likely due to them performing an important function in the protein or the genome itself. A suitable prototype genome in the context of the present disclosure is given in Enfissi et al. (supra). Nucleic acid sequences from Zika virus are available, e.g., in GenBank. Zika virus can be ordered from commercial sources, e.g., from the ATCC (see for instance Zika virus (ATCC® VR-84™) http://www.atcc.org/).

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

A “complementary” oligonucleotide, e.g., of a primer or probe corresponds to the antisense counterpart of a given oligonucleotide. That is, “A” is complementary to “T” and “G” to “C” and vice versa. Of course, this applies also to non-natural analogs of deoxynucleotides, as long as they are capable of “base-pairing” with their counterparts.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.” In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94° C.−96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time. Where the starting material for the PCR reaction is RNA, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe, thus increasing fluorescence with each replication cycle. SYBR® Green probes binds double-stranded DNA and upon excitation, emits light; thus, as PCR product accumulates, fluorescence increases. In the context of the present disclosure, the use of so-called TAQMAN® probes is preferred.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present disclosure, it is to be understood that the specific sequence lengths listed are illustrative and not limiting and that sequences covering the same map positions, but having slightly fewer or greater numbers of bases are deemed to be equivalents of the sequences and fall within the scope of the disclosure, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of 80%, 90%, 95%, 96%, 97%, 98%, or 99% or more fall within the scope of the present disclosure. As is known in the art, hybridization of complementary and partially complementary nucleic acid sequences may be obtained by adjustment of the hybridization conditions to increase or decrease stringency, i.e., by adjustment of hybridization temperature or salt content of the buffer. It is preferred that the sequence of the oligonucleotide is 99% and preferably 100% identical to the sequence of the oligonucleotides specified in the claims.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation. The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

Considering that Zika virus is an RNA virus the nucleic acid that is detected in the sample is typically RNA. Various amplification methods are available to the skilled person. Some of these are based on the polymerase chain reaction (PCR). These tests use a primer to rapidly make copies of the genetic material. A reverse transcriptase PCR (RT-PCR) is used to make the first copy to DNA for RNA viruses. Other methods such as nucleic acid sequence-based amplification (“NASBA”) can also be used in the present disclosure.

The kit of the disclosure or article of manufacture can also include a package insert having instructions thereon for using the primers, probes, and optional fluorophoric moieties to detect the presence or absence of Zika virus in a sample. In another aspect of the disclosure, there is provided a method for detecting the presence or absence of Zika virus in a biological sample from an individual. Such a method includes performing at least one cycling step. A cycling step includes at least one amplifying step and a hybridizing step. Generally, an amplifying step includes contacting the sample with a pair of primers to produce an amplification product if a Zika virus nucleic acid molecule is present in the sample. Generally, a hybridizing step includes contacting the sample with a Zika virus specific probe. The probe is usually labeled with at least one fluorescent moiety. The presence or absence of fluorescence is indicative of the presence or absence of Zika virus in the sample.

Amplification generally involves the use of a polymerase enzyme. Suitable enzymes are known in the art, e.g., Taq Polymerase, etc. In another aspect of the disclosure, there is provided a method for detecting the presence or absence of Zika virus in a biological sample from an individual. Such a method includes performing at least one cycling step. A cycling step can include an amplifying step and a dye-binding step. An amplifying step generally includes contacting the sample with a pair of Zika virus-specific primers of the disclosure to produce a Zika virus amplification product if Zika virus is present in the biological sample. A dye-binding step generally includes contacting the amplification products with a double-stranded DNA binding dye. The method further includes detecting the presence or absence of binding of the double-stranded DNA binding dye into the amplification product. According to the disclosure, the presence of binding is typically indicative of the presence of Zika virus nucleic acid in the sample, and the absence of binding is typically indicative of the absence of Zika virus nucleic acid in the sample. Such a method can further include the steps of determining the melting temperature between the amplification product and the double-stranded DNA binding dye. Generally, the melting temperature confirms the presence or absence of Zika virus in the sample. Representative double-stranded DNA binding dyes include SYBRGREEN I®, SYBRGOLD®, and ethidium bromide.

Oligonucleotides useful as amplification primers in the present disclosure are typically DNA. Oligonucleotides useful as probes are typically DNA comprising 0-4 modified nucleotides. In a preferred embodiment, the probe oligonucleotide comprises 0-4 locked nucleic acid nucleotides. A locked nucleic acid (LNA), often referred to as inaccessible DNA, is a modified DNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. Such oligomers are synthesized chemically and are commercially available. The locked ribose conformation enhances base stacking and backbone pre-organization. This significantly increases the hybridization properties (melting temperature) of oligonucleotides.

Amplification product in the context of the present disclosure refers to the nucleic acid that is the result of the amplification process. This typically contains the DNA sequence of the region of the Zika virus that is spanned by the oligonucleotide set used for the amplification flanked by the sequence of the oligonucleotides of the set used for the amplification.

The first oligonucleotide set comprises an oligonucleotide designated ON A and an oligonucleotide designated ON B. The second oligonucleotide set comprises an oligonucleotide designated ON C and an oligonucleotide designated ON D. The third oligonucleotide set comprises an oligonucleotide designated ON E and an oligonucleotide designated ON F.

ON A preferably comprises a consecutive stretch of at least 18 nucleotides of the sequence AARTACACATACCARAACAAAGTGGT (FP_ZIKA_San0-F) (SEQ ID NO:1) or a consecutive stretch of at least 18 nucleotides of the sequence TACACATACCARAACAAAGTGGT (FP_ZIKV_San0.alt-F) (SEQ ID NO:2).

ON B preferably comprises a consecutive stretch of at least 18 nucleotides of the sequence ACTTGTCCRCTCCCYCTYTGGTC (RP_ZIKA_San0-R) (SEQ ID NO:3).

ON C preferably comprises a consecutive stretch of at least 18 nucleotides of the sequence TGGTGTGGAAYAGRGTGTGGAT (FP_ZIKA_San-3F) (SEQ ID NO:4).

ON D preferably comprises a consecutive stretch of at least 18 nucleotides of the sequence GTAYTTYTCTTCATCACCTATG (RP_ZIKA_San-3R) (SEQ ID NO:5).

ON E preferably comprises a consecutive stretch of at least 18 nucleotides of the sequence GTTGTGGATGGAATAGTGGT (FP_ZIKA_San-1F) (SEQ ID NO:6).

ON F preferably comprises a consecutive stretch of at least 18 nucleotides of the sequence AGTARCACYTGTCCCATCT (RP_ZIKA_San-1R) (SEQ ID NO:7).

In a preferred embodiment, ON A comprises a consecutive stretch of 19, 20, 21, 22, 23, 24, 25 or 26 nucleotides of the sequence AARTACACATACCARAACAAAGTGGT (SEQ ID NO:1) or a consecutive stretch of 19, 20, 21, 22 or 23 nucleotides of the sequence TACACATACCARAACAAAGTGGT (SEQ ID NO:2). In a preferred embodiment, the ON A comprises a consecutive stretch of 20, 21, 22, 23, 24, 25 or 26 nucleotides of the sequence AARTACACATACCARAACAAAGTGGT (SEQ ID NO:1) or a consecutive stretch of 20, 21, 22 or 23 nucleotides of the sequence TACACATACCARAACAAAGTGGT (SEQ ID NO:2).

In a preferred embodiment, the ON A comprises a consecutive stretch of 22, 23, 24, 25 or 26 nucleotides of the sequence AARTACACATACCARAACAAAGTGGT (SEQ ID NO:1) or a consecutive stretch of 22 or 23 nucleotides of the sequence TACACATACCARAACAAAGTGGT (SEQ ID NO:2).

In a particularly preferred embodiment, the ON A comprises or consists of the sequence AARTACACATACCARAACAAAGTGGT (SEQ ID NO:1) or the sequence TACACATACCARAACAAAGTGGT (SEQ ID NO:2). ON A typically comprises either one or the other sequence. However, embodiments can be envisioned wherein ON A comprises two types of oligonucleotides; one comprising a sequence based on the sequence AARTACACATACCARAACAAAGTGGT (SEQ ID NO:1) and another comprising a sequence based on the sequence TACACATACCARAACAAAGTGGT (SEQ ID NO:2).

In a preferred embodiment, ON B comprises a consecutive stretch of 19, 20, 21, 22 or 23 nucleotides of the sequence ACTTGTCCRCTCCCYCTYTGGTC (SEQ ID NO:3). In a preferred embodiment, the ON B comprises a consecutive stretch of 20, 21, 22 or 23 nucleotides of the sequence ACTTGTCCRCTCCCYCTYTGGTC (SEQ ID NO:3). In a preferred embodiment, the ON B comprises a consecutive stretch of 21, 22 or 23 nucleotides of the sequence ACTTGTCCRCTCCCYCTYTGGTC (SEQ ID NO:3). In a particularly preferred embodiment, the ON B comprises or consists of the sequence ACTTGTCCRCTCCCYCTYTGGTC (SEQ ID NO:3).

In a preferred embodiment, ON C comprises a consecutive stretch of 19, 20, 21, or 22 nucleotides of the sequence TGGTGTGGAAYAGRGTGTGGAT (SEQ ID NO:4). In a preferred embodiment, the ON C comprises a consecutive stretch of 20, 21, or 22 nucleotides of the sequence TGGTGTGGAAYAGRGTGTGGAT (SEQ ID NO:4). In a preferred embodiment, the ON C comprises a consecutive stretch of 21, or 22 nucleotides of the sequence TGGTGTGGAAYAGRGTGTGGAT (SEQ ID NO:4). In a particularly preferred embodiment, the ON C comprises or consists of the sequence TGGTGTGGAAYAGRGTGTGGAT (SEQ ID NO:4).

In a preferred embodiment, ON D comprises a consecutive stretch of 19, 20, 21, or 22 nucleotides of the sequence GTAYTTYTCTTCATCACCTATG (SEQ ID NO:5). In a preferred embodiment, the ON D comprises a consecutive stretch of 20, 21, or 22 nucleotides of the sequence GTAYTTYTCTTCATCACCTATG (SEQ ID NO:5). In a preferred embodiment, the ON D comprises a consecutive stretch of 21, or 22 nucleotides of the sequence GTAYTTYTCTTCATCACCTATG (SEQ ID NO:5). In a particularly preferred embodiment, the ON D comprises or consists of the sequence GTAYTTYTCTTCATCACCTATG (SEQ ID NO:5).

In a preferred embodiment, ON E comprises a consecutive stretch of 18, 19 or 20 nucleotides of the sequence GTTGTGGATGGAATAGTGGT (SEQ ID NO:6). In a preferred embodiment, the ON E comprises a consecutive stretch of 19 or 20 nucleotides of the sequence GTTGTGGATGGAATAGTGGT (SEQ ID NO:6). In a preferred embodiment, the ON E comprises a consecutive stretch of 20 nucleotides of the sequence GTTGTGGATGGAATAGTGGT (SEQ ID NO:6). In a particularly preferred embodiment, the ON E comprises or consists of the sequence GTTGTGGATGGAATAGTGGT (SEQ ID NO:6).

In a preferred embodiment, ON F comprises a consecutive stretch of 17, 18 or 19 nucleotides of the sequence AGTARCACYTGTCCCATCT (SEQ ID NO:7). In a preferred embodiment, the ON F comprises a consecutive stretch of 18 or 19 nucleotides of the sequence AGTARCACYTGTCCCATCT (SEQ ID NO:7). In a preferred embodiment, the ON F comprises a consecutive stretch of 19 nucleotides of the sequence AGTARCACYTGTCCCATCT (SEQ ID NO:7). In a particularly preferred embodiment, the ON F comprises or consists of the sequence AGTARCACYTGTCCCATCT (SEQ ID NO:7).

In a preferred embodiment of the method of the disclosure, ON A comprises the sequence AARTACACATACCARAACAAAGTGGT (SEQ ID NO:1) or the sequence TACACATACCARAACAAAGTGGT (SEQ ID NO:2); ON B comprises the sequence ACTTGTCCRCTCCCYCTYTGGTC (SEQ ID NO:3); ON C comprises the sequence TGGTGTGGAAYAGRGTGTGGAT (SEQ ID NO:4); ON D comprises the sequence GTAYTTYTCTTCATCACCTATG (SEQ ID NO:5); ON E comprises the sequence GTTGTGGATGGAATAGTGGT (SEQ ID NO:6); and/or ON F comprises the sequence AGTARCACYTGTCCCATCT (SEQ ID NO:7).

In a preferred embodiment of the method of the disclosure, ON A comprises the sequence AARTACACATACCARAACAAAGTGGT (SEQ ID NO:1); ON B comprises the sequence ACTTGTCCRCTCCCYCTYTGGTC (SEQ ID NO:3); ON C comprises the sequence TGGTGTGGAAYAGRGTGTGGAT (SEQ ID NO:4) or TGTGGAAYAGRGTGTGGAT (nucleotides 4-22 of SEQ ID NO:4); ON D comprises the sequence GTAYTTYTCTTCATCACCTATG (SEQ ID NO:5); ON E comprises the sequence GTTGTGGATGGAATAGTGGT (SEQ ID NO:6); and ON F comprises the sequence AGTARCACYTGTCCCATCT (SEQ ID NO:7).

Where herein reference is made to an oligonucleotide sequence the reference is a base A, C, T or G. It is of course entirely possible to replace such a base with a base comprising the same base pairing capability in kind, not necessarily in amount.

The step of determining whether the sample comprises an amplification product preferably comprises incubating the sample during or after the amplification with one or more ZIKV specific probes that are specific for the amplification product of an amplification with the first oligonucleotide set, the second oligonucleotide set, the third oligonucleotide set or a combination of two or more of the sets.

The one or more probes preferably comprise an oligonucleotide with the sequence AAGGTYCTYAGACCA{G}{C}{T}{G}AA (SEQ ID NO:8); an oligonucleotide with the sequence CGCA{C}CA{C}HTGGG{C}TGA (SEQ ID NO:9); an oligonucleotide with the sequence AAAT{G}{G}ACA{G}ACATTCCCTA (SEQ ID NO:11); an oligonucleotide with the sequence ACTGACATTGACACAATGACAAT (SEQ ID NO:10); an oligonucleotide with the reverse complement of the sequence AAGGTYCTYAGACCA{G}{C}{T}{G}AA (SEQ ID NO:8); an oligonucleotide with the reverse complement of the sequence CGCA{C}CA{C}HTGGG{C}TGA (SEQ ID NO:9); an oligonucleotide with the reverse complement of the sequence AAAT{G}{G}ACA{G}ACATTCCCTA (SEQ ID NO:11); an oligonucleotide with the reverse complement of the sequence ACTGACATTGACACAATGACAAT (SEQ ID NO:10) or a combination of two or more of the oligonucleotides, wherein R=A or G; Y=C or T and H=A or T or C and wherein the nucleotides indicated with parenthesis are preferably locked nucleic acid nucleotides and wherein the one more probes comprise two or more of the oligonucleotides the two or more oligonucleotides preferably do not comprise oligonucleotides that are the reverse complement of each other.

The step of determining whether the sample comprises an amplification product preferably comprises monitoring amplification of the amplification product during the amplification process (real-time).

The disclosure also provides a kit or article of manufacture comprising one or more ZIKV specific probes that are specific for the amplification product of an amplification with the first oligonucleotide set, the second oligonucleotide set, the third oligonucleotide set or a combination of two or more of the sets. The one or more probes preferably comprise an oligonucleotide with the sequence AAGGTYCTYAGACCA{G}{C}{T}{G}AA (SEQ ID NO:8); an oligonucleotide with the sequence CGCA{C}CA{C}HTGGG{C}TGA (SEQ ID NO:9); an oligonucleotide with the sequence AAAT{G}{G}ACA{G}ACATTCCCTA (SEQ ID NO:11); an oligonucleotide with the reverse complement of the sequence AAGGTYCTYAGACCA{G}{C}{T}{G}AA (SEQ ID NO:8); an oligonucleotide with the reverse complement of the sequence CGCA{C}CA{C}HTGGG{C}TGA (SEQ ID NO:9); an oligonucleotide with the reverse complement of the sequence AAAT{G}{G}ACA{G}ACATTCCCTA (SEQ ID NO:11); or a combination the oligonucleotides, wherein R=A or G; Y=C or T and H=A or T or C and wherein the nucleotides indicated with parenthesis are preferably locked nucleic acid nucleotides and wherein the one more probes comprise two or more of the oligonucleotides the two or more oligonucleotides preferably do not comprise oligonucleotides that are the reverse complement of each other.

The probe with sequence AAGGTYCTYAGACCA{G}{C}{T}{G}AA (SEQ ID NO:8) detects amplification product obtained using the first oligonucleotide set. The probe with sequence CGCA{C}CA{C}HTGGG{C}TGA (SEQ ID NO:9) and the probe with the sequence AAAT{G}{G}ACA{G}ACATTCCCTA (SEQ ID NO:11) detect amplification product obtained using the second oligonucleotide set. The probe with sequence ACTGACATTGACACAATGACAAT (SEQ ID NO:10) detects amplification product obtained using the third oligonucleotide set. The same of course is true for the reverse complement of the oligonucleotides. It is, of course preferred to only use probes that are specific for a zika virus region that is amplified with a set of oligonucleotides. It typically serves no purpose to include a probe that is not specific for the amplified region in an amplification reaction as described herein.

Naturally, the appropriate probe is used. Thus, embodiments involving an oligonucleotide set for amplification and a probe, involve the set and the probe that can detect the amplification product produced by the set.

The kit or article of manufacture preferably comprises a first oligonucleotide set, a second oligonucleotide set, a third oligonucleotide set or a combination of two or more of the sets, wherein the first oligonucleotide set has ON A and ON B and the second set has ON C and ON D, the third set has ON E and ON F, wherein ON A, ON B, ON C, ON D, ON E and ON F are as defined elsewhere herein.

The disclosure also provides a kit comprising a first oligonucleotide set, a second oligonucleotide set, a third oligonucleotide set or a combination of two or more of the sets, wherein:

the first oligonucleotide set has:
an oligonucleotide ON A with a consecutive stretch of at least 18 nucleotides of the sequence AARTACACATACCARAACAAAGTGGT (SEQ ID NO:1) (FP_ZIKA_San0-F) or a consecutive stretch of at least 18 nucleotides of the sequence TACACATACCARAACAAAGTGGT (FP_ZIKV_San0.alt-F) (SEQ ID NO:2);

and
an oligonucleotide ON B with a consecutive stretch of at least 18 nucleotides of the sequence ACTTGTCCRCTCCCYCTYTGGTC (RP_ZIKA_San0-R) (SEQ ID NO:3);

the second oligonucleotide set has:
an oligonucleotide ON C with a consecutive stretch of at least 18 nucleotides of the sequence TGGTGTGGAAYAGRGTGTGGAT (FP_ZIKA_San-3F) (SEQ ID NO:4); and
an oligonucleotide ON D with a consecutive stretch of at least 18 nucleotides of the sequence GTAYTTYTCTTCATCACCTATG (RP_ZIKA_San-3R) (SEQ ID NO:5); and

the third oligonucleotide set has:
an oligonucleotide ON E with a consecutive stretch of at least 18 nucleotides of the sequence GTTGTGGATGGAATAGTGGT (FP_ZIKA_San-1F) (SEQ ID NO:6);
an oligonucleotide ON F with a consecutive stretch of at least 18 nucleotides of the sequence AGTARCACYTGTCCCATCT (RP_ZIKA_San-1R) (SEQ ID NO:7),

wherein R=A or G; Y=C or T.

The kit preferably comprises one or more Zika virus specific probes. The one or more ZIKV specific probes are specific for the amplification product of an amplification with the first oligonucleotide set, the second oligonucleotide set, the third oligonucleotide set or a combination of two or more of the sets. The one or more probes preferably comprise an oligonucleotide with the sequence AAGGTYCTYAGACCA{G}{C}{T}{G}AA (SEQ ID NO:8); an oligonucleotide with the sequence CGCA{C}CA{C}HTGGG{C}TGA (SEQ ID NO:9); an oligonucleotide with the sequence ACTGACATTGACACAATGACAAT (SEQ ID NO:10); an oligonucleotide with the sequence AAAT{G}{G}ACA{G}ACATTCCCTA (SEQ ID NO:11); an oligonucleotide with the reverse complement of the sequence AAGGTYCTYAGACCA{G}{C}{T}{G}AA (SEQ ID NO:8); an oligonucleotide with the reverse complement of the sequence CGCA{C}CA{C}HTGGG{C}TGA (SEQ ID NO:9); an oligonucleotide with the reverse complement of the sequence ACTGACATTGACACAATGACAAT (SEQ ID NO:10); an oligonucleotide with the reverse complement of the sequence AAAT{G}{G}ACA{G}ACATTCCCTA (SEQ ID NO:11); or a combination of two or more of the probe oligonucleotides, wherein R=A or G; Y=C or T and H=A or T or C and wherein the nucleotides indicated with parenthesis are preferably locked nucleic acid nucleotides and wherein the one more probes comprise two or more of the oligonucleotides the two or more oligonucleotides preferably do not comprise oligonucleotides that are the reverse complement of each other.

The disclosure further provides ON A, ON B, ON C, ON D, ON E, ON F or a combination thereof.

The disclosure further provides a composition comprising ON A and ON B.

The disclosure further provides a composition comprising ON C and ON D. The disclosure also provides a composition comprising ON E and ON F.

The disclosure further provides an apparatus arranged for performing an amplification of Zika virus nucleic acid comprising a biological sample to be tested for the presence of Zika virus nucleic acid, and one or more containers comprising:
an oligonucleotide ON A with a consecutive stretch of at least 18 nucleotides of the sequence AARTACACATACCARAACAAAGTGGT (FP_ZIKA_San0-F) (SEQ ID NO:1) or a consecutive stretch of at least 18 nucleotides of the sequence TACACATACCARAACAAAGTGGT (FP_ZIKV_San0.alt-F) (SEQ ID NO:2); and
an oligonucleotide ON B with a consecutive stretch of at least 18 nucleotides of the sequence ACTTGTCCRCTCCCYCTYTGGTC (RP_ZIKA_San0-R) (SEQ ID NO:3);

One or more containers comprising:
an oligonucleotide ON C with a consecutive stretch of at least 18 nucleotides of the sequence TGGTGTGGAAYAGRGTGTGGAT (FP_ZIKA_San-3F) (SEQ ID NO:4); and
an oligonucleotide ON D with a consecutive stretch of at least 18 nucleotides of the sequence GTAYTTYTCTTCATCACCTATG (RP_ZIKA_San-3R) (SEQ ID NO:5); and/or

One or more containers comprising:
an oligonucleotide ON E with a consecutive stretch of at least 18 nucleotides of the sequence GTTGTGGATGGAATAGTGGT (FP_ZIKA_San-1F) (SEQ ID NO:6); and
an oligonucleotide ON F with a consecutive stretch of at least 18 nucleotides of the sequence AGTARCACYTGTCCCATCT (RP_ZIKA_San-1R) (SEQ ID NO:7),

wherein R=A or G; Y=C or T.

In a preferred embodiment, the apparatus further comprises a container with the biological sample.

In a preferred embodiment, the apparatus further comprises one or more containers comprising one or more probe oligonucleotides with the sequence AAGGTYCTYAGACCA{G}{C}{T}{G}AA (SEQ ID NO:8); an oligonucleotide with the sequence CGCA{C}CA{C}HTGGG{C}TGA (SEQ ID NO:9); an oligonucleotide with the sequence ACTGACATTGACACAATGACAAT (SEQ ID NO:10); an oligonucleotide with the sequence AAAT{G}{G}ACA{G}ACATTCCCTA (SEQ ID NO:11); an oligonucleotide with the reverse complement of the sequence AAGGTYCTYAGACCA{G}{C}{T}{G}AA (SEQ ID NO:8); an oligonucleotide with the reverse complement of the sequence CGCA{C}CA{C}HTGGG{C}TGA (SEQ ID NO:9); an oligonucleotide with the reverse complement of the sequence ACTGACATTGACACAATGACAAT (SEQ ID NO:10); an oligonucleotide with the reverse complement of the sequence AAAT{G}{G}ACA{G}ACATTCCCTA (SEQ ID NO:11); or a combination of two or more of the oligonucleotides, wherein R=A or G; Y=C or T and H=A or T or C and wherein the nucleotides indicated with parenthesis are preferably locked nucleic acid nucleotides and wherein the one more probes comprise two or more of the oligonucleotides the two or more oligonucleotides preferably do not comprise oligonucleotides that are the reverse complement of each other.

A method for performing a Zika virus nucleic acid amplification reaction the method comprising providing an apparatus of the disclosure with a biological sample to be tested for the presence of Zika virus nucleic acid and allowing the apparatus to perform the amplification reaction.

Where herein a sequence of a particular nucleic acid molecule is depicted, the sequence is depicted in the 5′ to 3′ orientation.

Where herein reference is made to the letters R, Y or H in the context of a nucleotide sequence the letter stands for the nucleotides A and G when the letter is R, C and T when the letter is Y and A and T and C when the letter is H. The represented sequence is a sequence comprising one of the alternatives or a combination of two or more of the alternatives. An oligonucleotide comprising the sequence “GTAYTR” (SEQ ID NO:26) thus represents an oligonucleotide GTAATA (SEQ ID NO:27); GTAATG (SEQ ID NO:28); GTATTA (SEQ ID NO:29); GTATTG (SEQ ID NO:30); or a combination of two, three or four of the sequences.

In the present disclosure, it is preferred that the amplification method, kit, apparatus comprise or are performed with the first set of oligonucleotides and the second set of oligonucleotides. In the present disclosure, it is preferred that the amplification method, kit, apparatus comprise or are performed with the first set of oligonucleotides and the third set of oligonucleotides. In another embodiment, it is preferred that the amplification method, kit, apparatus comprise or are performed with the second set of oligonucleotides and the third set of oligonucleotides. A sample is positive for Zika virus nucleic acid if either one of the amplifications results in a detectable amplification product. The so-called dual target amplification significantly improves the performance of the method and reduces the number of false negative results as a result of sequence drift in the Zika virus in the test sample.

Oligonucleotides with a sequence that comprises a letter R, Y or H, as used in the present disclosure, typically are a mixture of oligonucleotides where some of the oligonucleotides in the mixture have one of the possible bases and other oligonucleotides in the mixture have another possible base. Where there are two alternative bases at one position the oligonucleotides in the mixture typically, but not necessarily, are 50% with one base at that position and 50% with the other.

### BRIEF DESCRIPTION OF THE DRAWINGS

FIG. 1: Schematic representation of the Zika virus genome and encoded proteins.

FIG. 2: Agarose gel (1%) electrophoresis result of the Zika virus PCR using the indicated primer sets.

FIGS. 3A and 3B: Real-time monitoring of amplification reaction on the LIGHTCYCLER® 480 instrument using primer set San0, San1, San3 and San4.

FIGS. 4A-4C: Real-time monitoring of amplification reaction on the cobas 6800 apparatus using San0, San 1, San3, San0/San3, San0/San1, and San1/San3 on a 10-fold dilution series diluted material of the Zika virus Tahiti material. The 10³-10¹⁰ times diluted samples were tested in duplicate.

### DETAILED DESCRIPTION

### Examples

A ZIKA virus amplification method has been developed and disclosed herein. The method was set up using the multi-channel utility of the Cobas 6800 apparatus from Roche. This channel is intended for the application of self-developed PCR testing using a Roche supplied by nucleic acid extraction and PCR reagents kit. The cobas 6800 is a high-throughput robot for automated nucleic acid extraction and PCR amplification. The ZIKV PCR can, among others be used for the screening of blood donors (for example, traveling blood donors).

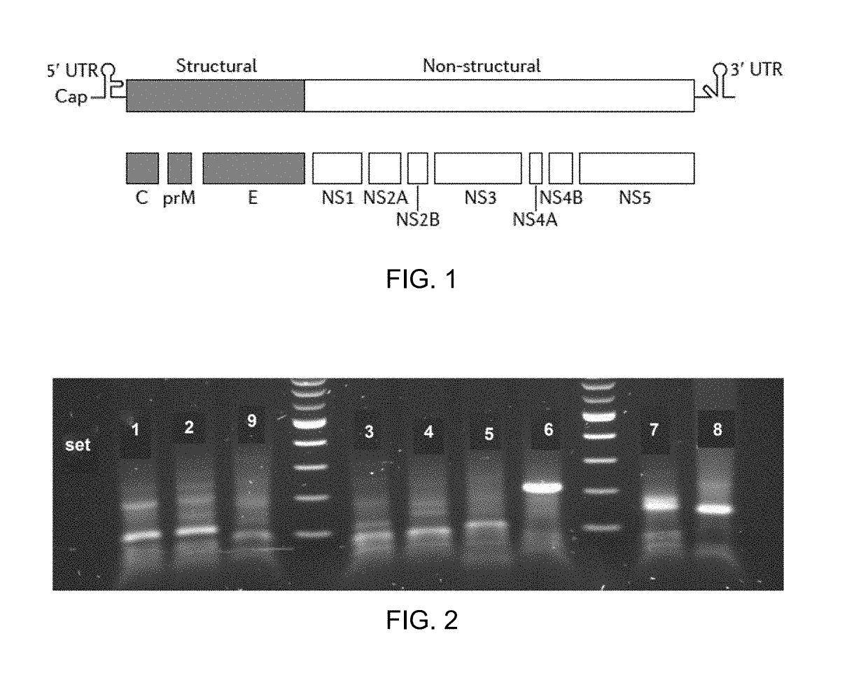

ZIKA virus is a member of the family of the Flaviviruses. The single-stranded RNA (+) genome, has a length of approximately 11,000 bases. The genome encodes a number of structural and nonstructural proteins (see FIG. 1) at both ends of the genome so-called untranslated regions (UTR). These UTRs include playing a role in the regulation of translation. UTRs are often GC-rich and usually contain many secondary structures (hairpin loops).

There are two strains of ZIKV known: the African tribe and the Asian strain. Phylogenetic studies have shown that epidemics in French Polynesia in 2014 and the epidemic in South America in 2015 are caused by the Asian strain of ZIKV.

On Feb. 4, 2016 in GenBank 28 “full length” taken from ZIKV known. The published length ranges from 9981 to 10,807 bases. In September 2015, at the time of setting up the ZIKA PCR, 24 “full length” taken from ZIKA were known.

### Example 1

### Initial PCR

Described are various PCR/detection strategies across the ZIKV genome (San0-San 5). Forward and reverse primers together with detections probes are listed in table 2.

Sample

For the testing of the ZIKV PCR test, use was made of ZIKV RNA-positive materials. This material Sanquin acquired from Dr. D. Musso (INSTITUT LOUIS MALARDE, Papeete-Tahiti-Polynésie française). Zika virus was isolated from the serum of a French Polynesian patient infected in 2013 (strain PF13/251013-18). The material is ZIKV containing supernatant from Zika virus propagated in African green Monkey kidney cells (Vero cells). In addition, ZIKV-positive culture medium (+/−10E8 cp/mL, inactivated) was obtained from the Bernhard-Nocht-Institut für Tropenmedizin, Hamburg, Germany. Both materials were used and showed no significant differences. The data obtained with the ZIKA material from Institut Louis Malarde, Papeete-Tahiti-Polynésie française) is shown in FIGS. 2-4C.

### Protocol Zika Virus PCR Reaction on LC480 Instrument

Nucleic acid extraction from plasma samples was performed with the EasyMag extractor (BioMerieux). The input volume for extraction was 1 mL. The elution volume was 50 μL. Ten μL was applied for single or dual target Zika virus PCR. The PCR was performed with the cobas Omni optimization kit (Roche Diagnostics, ordering number 07731663190) according to the instructions of the manufacturer. The final concentration of the primers was 500 nM of each primer. The final concentration of the probes was 125 nM each.

Pipetting Scheme

| | Single target | | Dual target |
|---|---|---|---|
| Reagents | Zika virus PCR | | Zika virus PCR |
| Water | 2 | μl | 0 |
| ZikaV Forward primer | 1 | μl | 1 μl primer 1 + 1 μl primer 2 |
| mix (20 μM) | | | |
| ZikaV Reverse primer | 1 | μl | 1 μl primer 1 + 1 μl primer 2 |
| mix (20 μM) | | | |
| ZikaV probe (5 μM) | 1 | μl | 1 μl probe 1 + 1 μl probe 2 |
| UC R1 Mn(Oac)2 | μl | | 10 μl |
| UC R2 Master mix | 15 | μl | 15 μl |
| Nucleic acid Extract | 10 | μl | 10 μl |
| Total PCR reaction | 40 | μl | 41 μl |
| volume | | | |

### Protocol Zika Virus PCR on Cobas 6800 Apparatus

The Cobas Omni Utility channel kit (Roche Diagnostics, ordering number 07557272190) and Cobas Buffer Negative control kit (Roche Diagnostics, ordering number 07002238190) were used as described by the manufacturer. The final concentration of the primers were 500 nM of each primer. The final concentration of the probes were 125 nM each.

The cobas 6800 (Roche Diagnostics) is a fully automated nucleic acid extraction and PCR apparatus.

### PCR Settings of the LC480 Instrument and the Cobas 6800 Apparatus

The PCRs were carried out on the LC480 instrument and cobas 6800 apparatus according to the following settings.

| | Number of | Temperature | |
|---|---|---|---|
| Step | cycles | (° C.) | Time (s) |
| Reverse | 1 | 55 | 120 |
| Transcription | | 60 | 360 |
| | | 65 | 240 |
| Pre-cycling | 5 | 95 | 5 |
| | | 55 | 30 |
| Cycling | 45 | 91 | 5 |
| | | 58 | 25* |
| Cooling | 1 | 40 | 10 |

*Measurement of fluorescent signal after this step

Testing of 9 Zika Virus Primer Sets with the Cobas Omni Optimization Kit on the LC480

The 9 primer sets were tested with cobas Omni optimization kit on the LC480 with extracts from cultured ZIKV material (10³×diluted Zika virus material from Bernhard-Nocht-Institut für Tropenmedizin, Hamburg, Germany).

Upon amplification the results of the amplification were visualized via agarose gel (1%) electrophoresis followed by SYBR® staining. The lanes were loaded as indicated in the table below. Unmarked lanes were loaded with a molecular size marker (100 bp DNA Ladder, New England Biolabs catnr: N3231S). San0, San1, San3 and San4 gave the best results based on the analyses on the agarose gel. The results of the electrophoresis are depicted in FIG. 2. All primer sets show a fragment of the expected size in the 9 lanes (see FIG. 2).

| | | | San0 | | | | San3- | | |
|---|---|---|---|---|---|---|---|---|---|
| | | | Alternative | | | | Alternative | | |
| | Faye | San0 | RP | San1 | San2 | San3 | FP | San4 | San5 |
| Forward | FP_ZIKV | FP_ZIKV | FP_ZIKV | FP_ZIKA_{—} | FP_ZIKA_{—} | FP_ZIKA_{—} | FP_ZIKA_{—} | FP_ZIKA_{—} | FP_ZIKA_{—} |
| primer | 9271-F | San-0F | San-0F | San-1F | San-2F | San-3Fa | San-3Fb | San-4F | San-5F |
| Reverse | RP_ZIKV | RP_ZIKA | RP_ZIKA_{—} | RP_ZIKA_{—} | RP_ZIKA_{—} | RP_ZIKA_{—} | RP_ZIKA_{—} | RP_ZIKA_{—} | RP_ZIKA_{—} |
| primer | 9352-R | San-0R | San0-AltB | San-1R | San-2R | San-3R | San-3R | San-4R | San-5R |
| Lane | 1 | 2 | 3 | 4 | 5 | 6 | 7 | 8 | 9 |
| Size (bp) | 102 | 107 | 80 | 83 | 96 | 209 | 147 | 140 | 80 |
| Result | | Nr. 2 | | | | Nr. 1 | | Nr. 3 | |

### San0, San1, San3 and San4 PCR on the LIGHTCYCLER® 480 (LC480) PCR Instrument

The selected three primer sets (San0, San1, San3 and San4) were tested with the indicated probes on 100 cp/ml Zika or dilution series ranging from 10⁵ to 10 copies/mL Zika virus RNA (Zika virus material from Germany and Zika virus material from Tahiti). The PCRs were performed with cobas Omni optimization kit reagents on the LC480.

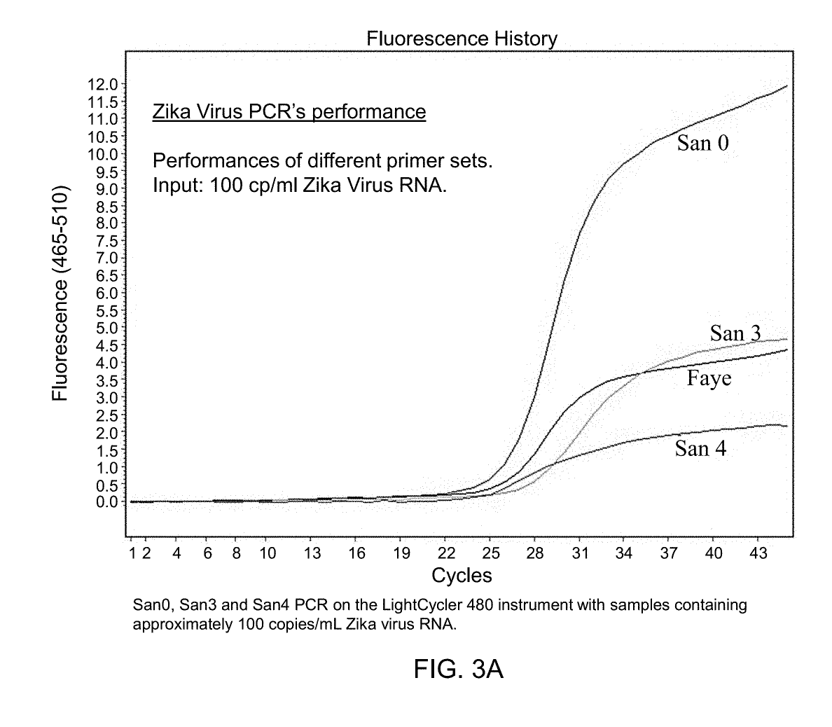

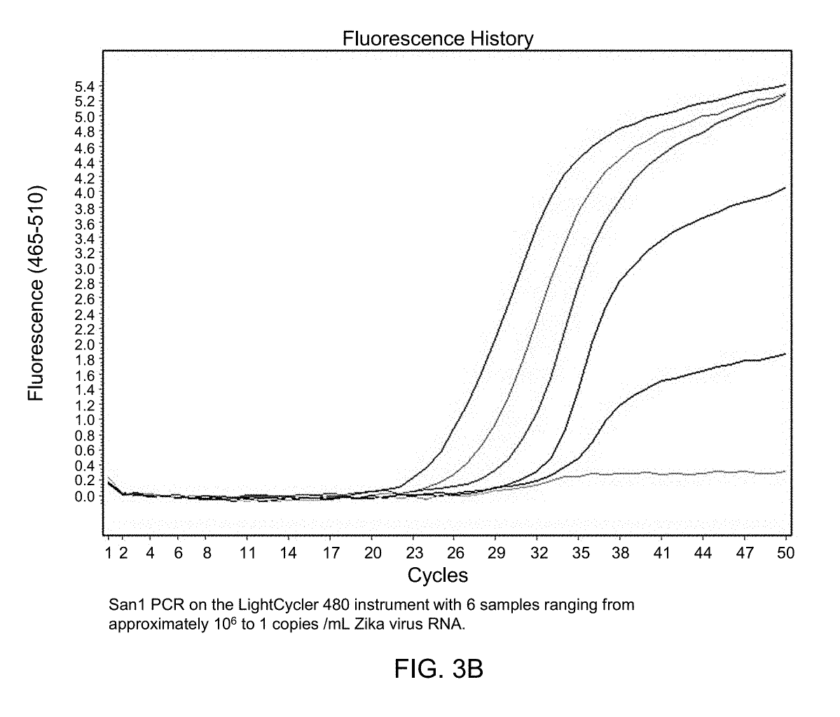

The results are shown in FIGS. 3A and 3B. San4 gave poor fluorescent signals. Therefore, we proceeded with San0, San1 and San3 giving good fluorescent signals. For San0, the initial runs were done with the longest forward primer (FP_ZIKV_San-0F). We also tested the shorter one (FP_ZIKV_San-0F-alt). We did not notice significant differences between both forward primers. For San3 we started with the long probe (Probe_ZIKA_San-3P) with three wobble bases. We also tested a shorter one (Probe_ZIKA_San-3Pturbo) with three LNA bases. We did not notice significant differences between both probes.

San0, San1 and San3 were tested in combination with the selected probes on the LIGHTCYCLER® 480 (Roche) with the cobas Omni optimization kit. Next, the San0, San1, and San3 and the combination San0/San3, San0/San1 and San1/San3 were tested on the utility channel of the cobas 6800.

### Dual-Target PCR on Cobas 6800

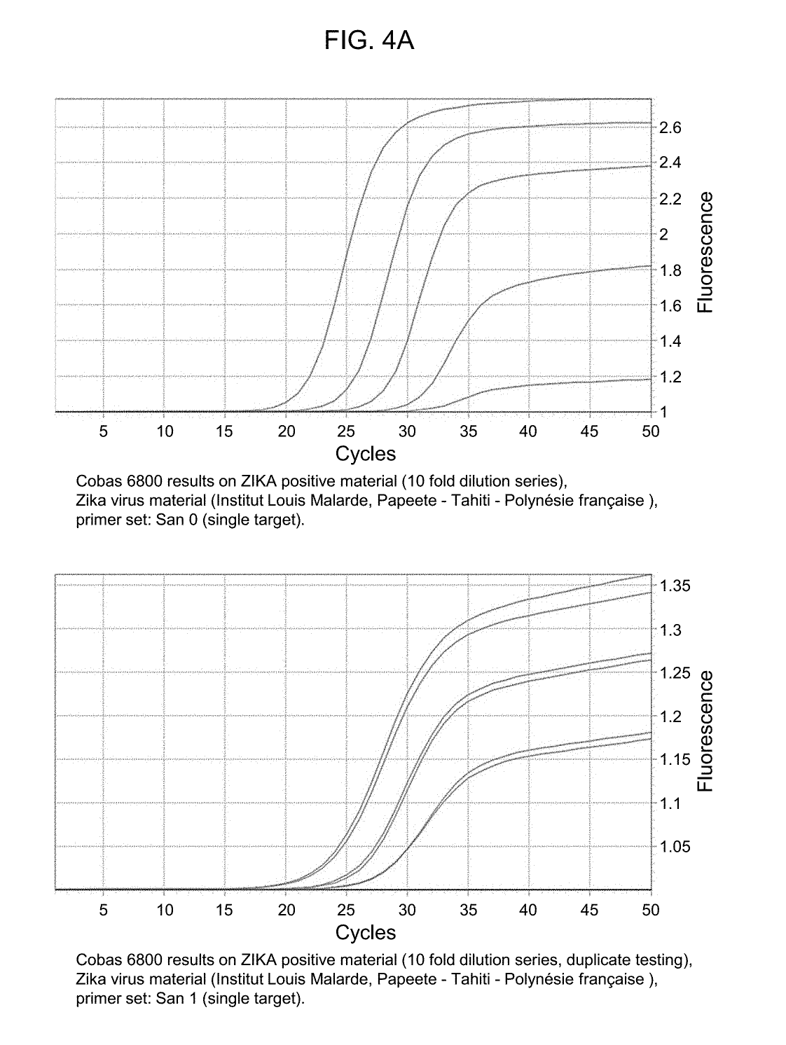

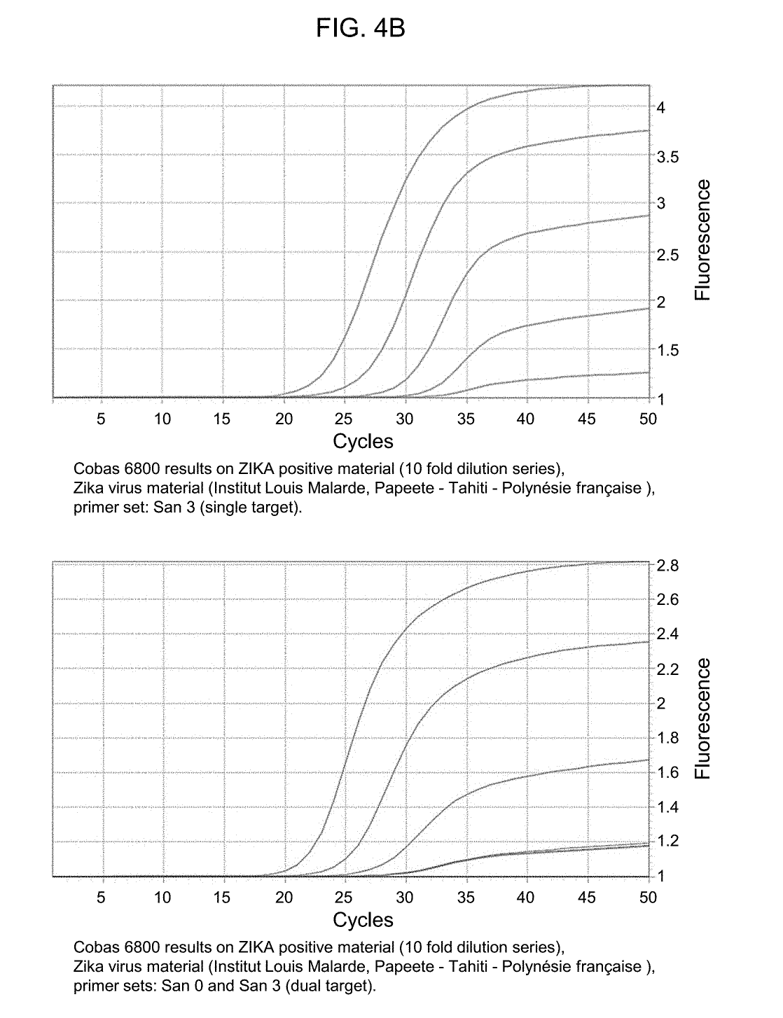

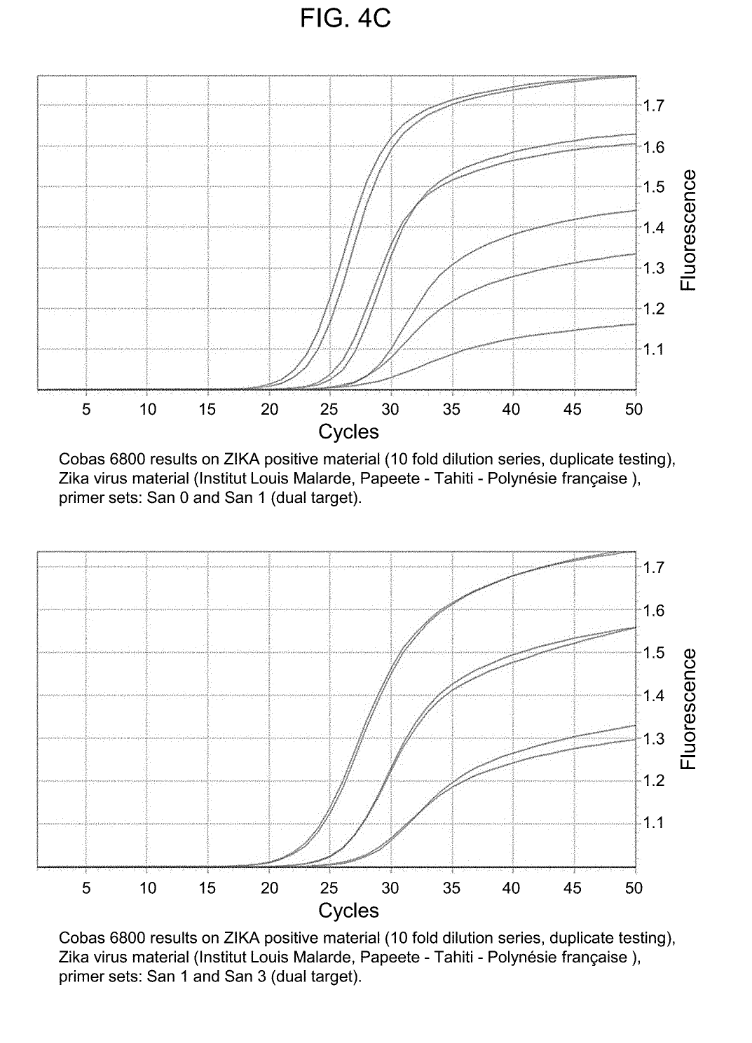

Results of testing San0, San1, and San3 and the combination San0/San3, San0/San1 and San1/San3 primer sets on the multi-channel utility of the cobas 6800 are shown in FIGS. 4A-4C. It can be concluded that:
All selected primer sets work on the Utility channel of the cobas 6800 apparatus based on 10-fold dilution series of Zika-positive material.
Dual target PCR with San0/San3, San0/San1 and San1/San3 primer sets primer/probe sets works on the Utility channel of the cobas 6800 apparatus based on 10-fold dilution series of Zika-positive material.

Again pipetting scheme, concentration of the relevant ingredients (primers, virus, probe) see above. The same pipetting scheme is used in all experiments.

### Example 2

### Materials and Methods

### Primer and Probe Design

The ZIKV PCR assay targets conserved sequences in the 3′-UTR region of the ZIKV genome. Using Geneious software (version 7.1), 69 complete African and Asian ZIKV genomic sequences, available in the Genbank databases on 30 Oct. 2015, were used for primer and probe design. The selected primer set (purchased from Invitrogen Life Technologies, Cergy Pontoise, France) amplifies a 206 base pair fragment. The PCR product was verified on agarose gels (data not shown). To improve the detection of genetic variants of ZIKV, two probes (purchased from Eurogentec S.A. Seraing, Belgium) were selected. One hybridizes to the template and covers the Asian lineage. The other probe hybridizes to the complementary strand and covers the African lineage. Primer and probe sequences are shown in Table 3. On 20 Dec. 2016, the PCR primers and probes potential mismatches were evaluated by mapping them to ZIKV full-length sequences available in Genbank. Twenty-nine were of the African and 176 were of the Asian ZIKV lineage. The primer set in combination with the probe covering the Asian lineage showed up to five potential mismatches in 176 Asian isolates. The primer set in combination with the probe covering the African lineage showed up to maximal four potential mismatches in 29 African isolates. No mismatches were found near the 3′-end of the primers, which is crucial for detection through PCR.

### Optimization of PCR Conditions

The optimum primer/probe concentrations and the PCR cycling condition were determined on the LIGHTCYCLER® 480-II instrument (Roche, Pleasanton, Calif., USA). Viral RNA was extracted using the NUCLISENS EASYMAG® extractor (bioMérieux, Marcy-l'Étoile, France) with the NUCLISENS® magnetic extraction reagents according to the off-board protocol, which is based on the method developed by Boom et al. (J. Clin. Microbiol. 1990; 28: 495-503). Briefly, 850 μL plasma was mixed with 9 mL lysis buffer containing 6 M guanidine isothiocyanate (GIT). To monitor extraction and PCR amplification efficiency, 10 μL of noncompetitive internal control (IC) molecules (MS2 bacteriophage, DSMZ, Braunschweig, Germany) was added, which results in IC signals with Ct values between 35-40 cycles (Dreier et al., J. Clin. Microbiol. 2005; 43: 4551-7). The lysis buffer tubes were incubated at 37° C. for 30 minutes. After addition of 50 μL magnetic silica particles, tubes were incubated at room temperature for 10 minutes. The silica particles were pelleted by centrifugation at 3000 rpm for 5 minutes. Next, the pellet was resuspended in 1.8 mL of lysis buffer and transferred to EASYMAG® vessels. Nucleic acid was eluted in 50 μL elution buffer. PCR was performed on the LIGHTCYCLER® 48041 using the cobas omni Optimization Kit (Roche, Pleasanton, Calif., USA). The cobas omni Optimization Kit contains the same reagents as present in the reagent cassette of the cobas omni UC on the cobas 6800 System. The total volume of one PCR reaction was 52 μL and consisted of 15 μL Master Mix Reagent 2 (MMx-R2), 10 μL manganese acetate (MMx-R1), and 27 μL nucleic acid. The optimal primers and probe concentrations were 500 nM for each primer and 80 nM for each probe (data not shown). The RT-PCR protocol consisted of reverse transcription for 7 minutes at 55° C. and 7 minutes at 65° C., followed by 50 cycles of 5 seconds at 91° C. and 40 seconds at 60° C.

### ZIKV PCR Test on the Cobas Omni Utility Channel on the Cobas 6800 System

The UC on the cobas 6800 System is an open channel that utilizes a pre-assembled reagent cassette consisting of five containers including one empty container. The reagent cassette was prepared according to the manufacturer's instructions. Four containers contain protease solution, internal control solution, elution buffer and MMx-R1 (manganese acetate), respectively. MMx-R2 was added in the empty container. MMx-R2 was prepared as follows: eight mL of the cobas omni UC MMx-R2, which includes primers and probe for amplification and detection of the internal control, was mixed gently for 5 minutes. Subsequently, 140 μL of each primer (100 μM), 22 μL of each probe (100 μM) and 156 μL H₂O was added and mixed for another 5 minutes. Finally, 7 mL of the prepared master mix was transferred to the empty container of a cobas omni UC Reagent Kit cassette.

The cobas omni Utility Channel Tool software version 1.0 (Roche, Pleasanton, Calif., USA) was used to put the PCR profile, sample type/input volume and Relative Fluorescence Increase (RFI value) cut-off value on the cobas 6800 System. The sample input volume and the RFI were set on 850 μL and 1.07 respectively. The cobas omni Utility Channel Tool software was also used to label the assay name on the cassette radio-frequency identification tag, which enables the cassette to be identified by the cobas 6800 System.

### Analytical Sensitivity

The analytical sensitivity was determined by testing 0.5 log dilution series of the WHO international standard for ZIKV RNA (WHO ZIKV IS) for NAT assays (11468/16, Paul-Ehrlich-Institute, Langen, Germany) (Baylis et al, Zika Virus Collaborative Study G. Harmonization of nucleic acid testing for Zika virus: development of the 1st World Health Organization International Standard. Transfusion 2017). The dilutions were prepared in negative pooled plasma and in urine (from one individual). The WHO ZIKV IS is a heat-inactivated and lyophilized cell culture-derived preparation from the French Polynesian ZIKV strain (Asian lineage). The assigned unitage is 5×10⁷ IU/mL. The WHO ZIKV IS was diluted in negative human plasma in half-log increments to concentrations spanning the previously determined end-point. The dilutions consisted of five concentrations ranging from 50 to 0.5 IU/mL and were tested in two independent sets of 24 replicates per dilution (total 48 replicates). For urine one set of twelve replicates per dilution was tested. The plasma or urine input volume was 850 μL. The 95% Limit of detection (LOD) was determined by Probit regression analysis using IBM SPSS Statistics version 23 with ¹⁰log converted input concentrations.

### Clinical Sensitivity

The 29 clinical plasma samples used in this study were from the Medical Laboratory Services (MLS), Willemstad, Curaçao. The plasma samples were derived from individuals suspected of ZIKV infection by the general practitioner. The samples were shipped to Sanquin and kept at <−20° C. until processing. At Sanquin, the samples were tested with the ZIKV PCR and with the investigational cobas Zika test (Roche, Pleasanton, Calif., USA). The cobas Zika test was developed for the detection of ZIKV RNA in blood donor plasma samples using the cobas 6800/8800 Systems and was recently authorized by the Food and Drug Administration, under an investigational new drug application, for ZIKV donor screening in the United States (Kuehnert et al., Screening of Blood Donations for Zika Virus Infection—Puerto Rico, Apr. 3-Jun. 11, 2016; MMWR Morb. Mortal Wkly. Rep. 2016; 65: 627-8; Galel et al., First Zika-positive donations in the continental United States, Transfusion 2017). Due to limited sample volume, 0.2 mL plasma of each sample was first diluted in 1.8 mL negative human plasma. The 10× diluted samples were tested with both ZIKA assays on the cobas 6800 System. The input volume was 850 μL per sample for both assays.

### Specificity of the ZIKV PCR on the Cobas 6800

The specificity of the primer/probe set for the ZIKV PCR assay was evaluated by testing of 186 ZIKV-negative individual Dutch blood donor samples (healthy blood donors from anon-endemic area). In addition, the potential cross-reactivity with other blood transmittable and arboviruses was evaluated by testing sets of the following plasma samples and concentration ranges: 5×HCV samples (4×10⁶-1×10⁷ IU/mL), 5× hepatitis E virus samples (3×10⁴-4×10⁴ IU/mL), 5×HIV samples (3×10⁵-1×10⁶ IU/mL), 5×HBV samples (5×10⁶-2×10⁸ IU/mL) and 5× parvovirus B19 samples (1×10⁷-2×10¹⁰ IU/mL); 4× Dengue virus (DENV) samples with a concentration of 1×10⁷ copies/mL, each sample representing one of the four DENV genotypes; 2× West-Nile virus samples (lineage 1 and lineage 2) with a concentration of 1×10⁸ copies/mL; 1× Chikungunya virus sample with a load of 1×10⁷ copies/mL and 1× hepatitis A virus sample with a load of 1×10⁴ IU/mL.

To ensure the ubiquity of the designed primer/probe set to detect African and Asian ZIKV strains, different ZIKV isolates from both lineages were tested. The testing included two African ZIKV strain isolated from Uganda, MR 766 and MP 1751 (Dick et al., Transactions of The Royal Society of Tropical Medicine and Hygiene 1952, 46: 509-20; Haddow et al., Bulletin of the World Health Organization 1964, 31: 57-69). The ZikaSPH2015 strain from Brazil and PF13/251013-18 (the WHO ZIKV IS) isolated from French Polynesia representing the Asian ZIKV lineage (Cunha et al., Genome Announcements 2016; 4: e00032-16; Trosemeier et al., Genome Announcements 2016; 4: e00917-16).

### Results

### Analytical Sensitivity

The Limit of Detection (LOD) of the ZIKV PCR assay was determined by analyzing serial dilutions in plasma or urine of the candidate WHO ZIKV IS on the cobas omni UC of the cobas 6800 System. The number and percentage of positive results of each concentration for 48 replicates is shown in Table 4. The data in this table were used for Probit analyses. For plasma, the ZIKV PCR assay demonstrated a 95% LOD of 23.0 IU/mL (95% confidence interval: 16.5-37.5). The 50% hit rate was 5.3 IU/mL (95% confidence interval: 4.3-6.6).

For urine, The ZIKV PCR assay demonstrated a 95% LOD of 24.5 IU/mL (95% confidence interval: 13.4-92.9). The 50% hit rate was 5.4 IU/mL (95% confidence interval: 3.3-8.8).

### Clinical Sensitivity

The 29 clinical samples obtained from suspected naturally infected individuals were analyzed simultaneously with the ZIKV PCR and the Roche cobas Zika test for use on the cobas 6800/8800 Systems. The results are shown in Table 5. A total of 13/29 (45%) were reactive with the ZIKV PCR assay and the cobas Zika test. Furthermore, 11/29 samples (38%) were non-reactive in both PCR assays.

Five samples (17%) showed discrepant results: two samples were only reactive with the ZIKV PCR assay and three samples were only reactive with the cobas Zika test. The discrepant samples showed high Ct values (>37.4) indicating very low ZIKV RNA loads. At low ZIKV RNA levels discrepancies can be expected. Due to insufficient sample volume, it was not possible to retest the discrepant samples.

### Specificity

The ZIKV PCR assay showed a specificity of 100% when testing 186 ZIKV-negative blood donor samples. All 186 samples were non-reactive in the ZIKV PCR assay and showed a valid IC signal. The average Ct value of the IC curves was 31.14 cycles (SD=0.4 cycles).

Potential cross-reactivity of the ZIKV PCR assay was evaluated by testing samples containing other viruses. The ZIKV PCR assay on the cobas omni UC on the cobas 6800 System showed no cross-reactivity with CHIKV, DENY, HBV, HCV HEV HIV, parvovirus B19 and WNV. All samples showed a valid IC signal.

All ZIKV isolates tested in this study were reactive in the ZIKV PCR assay. The Ct value of the samples ranged from 34.2 to 36.6. These findings indicate that the assay is able to detect strains from both the African and Asian ZIKV lineage.

### DISCUSSION

The ZIKV PCR assay LOD was not significantly different in plasma and urine as determined on testing serial dilutions of the candidate ZIKV IS. The ZIKV PCR assay LOD was 23.0 IU/mL (95% CI: 16.5-37.5) in plasma and 24.5 IU/mL (95% CI: 13.4-92.9) in urine. This is the first study using the WHO ZIKV IS. Therefore, it is difficult to compare our assay with previously published ZIKV PCR assays. However, a recent study compared nine published ZIKV PCR assays using a synthetic universal control ribonucleic acid construct containing the target regions of each assay (V. M. Corman et al., Bulletin of the World Health Organization 2016, 94: 880-92). Extrapolation of the findings of this study to viral loads and accommodating the loads to our input and extraction volume, the LOD of eight assays out of nine ranged between 20 to 170 copies/mL. The ninth assay had an extrapolated LOD of 13,800 copies/mL. Assuming that one copy is equivalent to one IU, our ZIKV PCR assay shows comparable sensitivity with most ZIKV PCR assays included in this comparative study. The availability of the WHO ZIKV IS will allow a much better comparison of the sensitivity of the available ZIKV PCR assays.

The ZIKV PCR assay was highly specific and showed no-cross reactivity with several other viruses. Moreover, all subjected ZIKV strains in this study were reactive, indicating that the assay detect sequence variants of African and Asian ZIKV lineages (Dick et al., Transactions of The Royal Society of Tropical Medicine and Hygiene 1952, 46: 509-20; Haddow et al., Bulletin of the World Health Organization 1964, 31: 57-69; Cunha et al., Genome Announcements 2016, 4: e00032-16; and Trosemeier et al., Genome Announcements 2016, 4: e00917-16). The applied primers target a conserved part of the 3′-UTR region. The potential mismatches were not located at the 3′-end of the primer. In addition, to anticipate future ZIKV variants the assay is designed with two probes, which will permit ZIKV detection in case of new nucleotide mismatches. When the assay was performed with only one of the probes the assay still had approximately the same sensitivity and could detect strains from both lineages (data not shown).

The evaluation of the 29 clinical samples shows that the ZIKV PCR assay was in 83% of the cases concordant with the investigational Roche cobas Zika test. The reason for the five discrepancies remains uncertain. The relatively high Ct values of the discrepant reactive results are indicative of a low sample ZIKV RNA load, which could be an explanation for the observed discrepancies. The information concerning the time between onset of symptoms and sample collection was not available. Therefore, the eleven negative results could be a result of patients not having a ZIKV infection, or due to ZIKV RNA clearance by the patient. The non-reactive result could also be explained by the low viral load as a consequence of 10×pre-dilution of the samples before testing. As mentioned, due to the limited sample amount it was not possible to retest the samples.

Sanquin applies a 28-day deferral period for donors returning from a travel outside of Europe to prevent donation by donors with asymptomatic viremia of known and unknown infectious diseases. Donors travelling within Europe are not deferred unless they have traveled to an area determined by the Dutch blood transfusion service as risk area for infectious diseases. This precaution measure was recently implemented during the WNV outbreak, which affected parts of Europe. Although this deferral policy is cost-effective, safe and can be implemented rapidly, it can have a significant impact on donor availability, especially after the holiday seasons (Lieshout-Krikke et al., Transfusion 2015, 55: 79-85; Lieshout-Krikke et al., Vox Sang 2013, 104: 12-8). Therefore, in the future, it might be necessary to selectively test donors with a recent travel history with a NAT for the endemic viruses and release the blood when the result is non-reactive. This may also apply for countries endemic for ZIKV. Following the WNV outbreak, some European countries have introduced a selectively WNV NAT testing based on travel history (Pisani et al., Transfusion Medicine and Hemotherapy 2016, 43: 158-67). The risk of a ZIKV outbreak in Europe is feasible since one of its vectors, Ae. albopictus, is present in South-Europe and ZIKV imported cases into Europe have been reported (Massad et al., Global Health Action 2016, 9: 10.3402/gha.v9.31669). In case of a ZIKV outbreak in Europe, the ZIKV PCR can be used for selective ZIKV testing of travelling donors to avoid deferral. Selective testing of donors returning from affected regions is an alternative, in case deferral leads to an unacceptable decrease of donor availability.

WHO recently published a target profile for ZIKV PCR assays and one of the desired characteristics for an optimal ZIKV diagnostic test is a multiplex PCR of these three arboviruses (WHO, Target Product Profiles for better diagnostic tests for Zika Virus Infection 2016). Ideally, an arbo-PCR multiplex also includes WNV. Recently, three multiplex assays targeting ZIKV, DENV and CHIKV have been published, including one released by CDC under an emergency authorization (Waggoner et al., Emerg. Infect. Dis. 2016, 22: 1295-7; Calvo et al., Acta Trop 2016, 163: 32-7; and Pabbaraju et al., J. Clin. Virol. 2016, 83: 66-71). None of the three multiplex PCRs was evaluated with the cobas omni UC reagents on the cobas 6800 System. Partially or complete adoption and/or modification of the primer sets used by one of three published multiplex PCRs could be an alternative for a new primer design covering DENV and CHIKV.

TABLE 1
Zika virus primers and probes

| Set | Name | 5′ to 3′ sequence | Remark |
|---|---|---|---|
| San- | FP_ZIKA_ | AARTACACATACCARAACAAAGTGGT | NS5 |
| 0 | San0-F | (SEQ ID NO: 1) | region |
| | RP_ZIKA_ | ACTTGTCCRCTCCCYCTYTGGTC | |
| | San0-R | (SEQ ID NO: 3) | |
| | Probe_ | FAM- | {nt} = |
| | ZIKA- | AAGGTYCTYAGACCA{G}{C} | LNA |
| | San0-P | {T}{G}AA-BHQ1 | nucleo- |
| | | (SEQ ID NO: 8) | tide |
| * | FP_ZIKV_ | TACACATACCARAACAAAGTGGT | * = |
| | San0.alt- | (SEQ ID NO: 2) | Alter- |
| | F | | native |
| | | | San-0 |
| | | | forward |
| | | | primer |
| San- | FP_ZIKA_ | GTTGTGGATGGAATAGTGGT | NS4B |
| 1 | San-1F | (SEQ ID NO: 6) | region |
| | RP_ZIKA_ | AGTARCACYTGTCCCATCT | |
| | San-1R | (SEQ ID NO: 7) | |
| | Probe_ | FAM- | |
| | ZIKA_ | ACTGACATTGACACAATGACAAT- | |
| | San-1P | BHQ1 (SEQ ID NO: 10) | |
| San- | FP_ZIKA_ | TGGTGTGGAAYAGRGTGTGGAT | 3′-UTR |
| 3 | San-3F | (SEQ ID NO: 4) | region |
| | RP_ZIKA_ | GTAYTTYTCTTCATCACCTATG | |
| | San-3R | (SEQ ID NO: 5) | |
| | Probe_ | FAM-CGCA{C}CA{C}HTGGG | {nt} = |
| | ZIKA_ | {C}TGA-BHQ1 | LNA |
| | San-3P | (SEQ ID NO: 9) | nucleo- |
| | | | tide |

TABLE 2

| Set | Name | 5′ to 3′ sequence | Remark |
|---|---|---|---|
| Faye* | FP_ZIKV | AARTACACATACCARAACAAAGTGGT | |
| | 9271-F | (SEQ ID NO: 1) | |
| | RP_ZIKV | TCCRCTCCCYCTYTGGTCTTG | |
| | 9352-R | (SEQ ID NO: 12) | |
| | Probe | CTYAGACCA{G}{C}{T}{G}AAR | {nt} = |
| | ZIKV | (SEQ ID NO: 13) | LNA |
| | 9304-P | | nucleo- |
| | | | tide |
| | | | (FAM/ |
| | | | BBQ) |
| San 0 | FP_ZIKV | AARTACACATACCARAACAAAGTGGT | |
| | San-0F | (SEQ ID NO: 1) | |
| | FP_ZIKV | TACACATACCARAACAAAGTGGT | |
| | San-0F- | (SEQ ID NO: 2) | |
| | alt | | |
| | RP_ZIKA | ACTTGTCCRCTCCCYCTYTGGTC | |
| | San-0R | (SEQ ID NO: 3) | |
| | RP_ZIKA_ | CTTGARATRATGTCCATRACTGT | |
| | San0-AltB | (SEQ ID NO: 14) | |
| | Probe_ | AAGGTYCTYAGACCA{G}{C} | {nt} = |
| | ZIKA_ | {T}{G}AA | LNA |
| | San0P | (SEQ ID NO: 8) | nucleo- |
| | | | tide |
| | | | (FAM/ |
| | | | BHQ1) |
| San 1 | FP_ZIKA | GTTGTGGATGGAATAGTGGT | |
| | San-1F | (SEQ ID NO: 6) | |
| | RP_ZIKA_ | AGTARCACYTGTCCCATCT | |
| | San-1R | (SEQ ID NO: 7) | |
| | Probe_ | ACTGACATTGACACAATGACAAT | FAM/ |
| | ZIKA_San- | (SEQ ID NO: 10) | BHQ1 |
| | 1P | | |
| San 2 | FP_ZIKA_ | GAYGGKAGRTCCATTGTGGT | |
| | San-2F | (SEQ ID NO: 15) | |
| | RP_ZIKA_ | AGTCTCMCGRATGCTCCATCC | |
| | San-2R | (SEQ ID NO: 16) | |
| | Probe_ | CGCCACCAAGATGAAYTGATTGGCCG | |
| | ZIKA_San- | (SEQ ID NO: 17) | |
| | 2P | | |
| San 3 | FP_ZIKA_ | TGGTGTGGAAYAGRGTGTGGAT | |
| | San-3Fa | (SEQ ID NO: 4) | |
| | FP_ZIKA_ | AARTGGACAGAHATYCCCTA | |
| | San-3Fb | (SEQ ID NO: 18) | |
| | RP_ZIKA_ | GTAYTTYTCTTCATCACCTATG | |
| | San-3R | (SEQ ID NO: 5) | |
| | Probe_ | ATA GGG CAC AGR CCN CGC | FAM/ |
| | ZIKA_San- | ACY AC | BHQ1 |
| | 3P | (SEQ ID NO: 19) | |
| | Probe_ | CGCA{C}CA{C}HTGGG{C}TGA | {nt} = |
| | ZIKA_San- | (SEQ ID NO: 9) | LNA |
| | 3Pturbo | | nucleo- |
| | | | tide |
| | | | (FAM/ |
| | | | BHQ1) |
| San 4 | FP_ZIKA_ | TTAGCTGGKCCACTCAG | |
| | San-4F | (SEQ ID NO: 20) | |
| | RP_ZIKA_ | GTTCCRCATGTYTCCTCCAC | |
| | San-4R | (SEQ ID NO: 21) | |
| | Probe | AGTGAAGAGCTYGAAATYCGGTT | FAM/ |
| | ZIKA San- | TGAGGA | BHQ1 |
| | 4P | (SEQ ID NO: 22) | |
| San 5 | FP_ZIKA_ | GTGGAGGARACATGYGGAAC | |
| | San-5F | (SEQ ID NO: 23) | |
| | RP_ZIKA_ | CAGCACCATTCCTCDATSAC | |
| | San-5R | (SEQ ID NO: 24) | |
| | Probe_ | GGAAGGGTSATHGAGGAATGGTGCT | |
| | ZIKA_San- | (SEQ ID NO: 25) | |
| | 5P | | |

*Faye et al., Virol. J. 2013 Oct 22; 10:311

TABLE 3

| | Genome | |
|---|---|---|
| Name | position* | Sequence (5′ → 3′) |
| ZIKA- | 10,091-10,109 | TGTGGAACAGAGTGTGGAT |
| Forward | | (nucleotides 4-22 |
| | | of SEQ ID NO: 4 |
| | | with nucleotide 11 |
| | | chosen as C and 14 |
| | | chosen as A) |
| ZIKA- | 10,275-10,296 | GTACTTTTCTTCATCACCTATG |
| Reverse | | (SEQ ID NO: 5 with |
| | | nucleotide 4 chosen as |
| | | C and 7 chosen as T) |
| ZIKA | 10,150-10,169 | [FAM]- |
| probe- | | AAAT{G}{G}ACA{G} |
| Forward | | ACATTCCCTA-[BHQ1] |
| | | (SEQ ID NO: 11) |
| ZIKA | 10,222-10,238 | [FAM]-TCA{G}CCCAD{G} |
| probe- | | TG{G}TGCG-[BHQ1] |
| Reverse | | (SEQ ID NO: 31) |

*Genome positions based on KX369547 in Genbank (sequence of candidate International ZIKV standard)
{G} = LNA base

TABLE 4

| Concen- | Plasma | | | Urine | | |
|---|---|---|---|---|---|---|
| tration | N- | N- | % | N- | N- | % |
| IU/mL | tested | Reactive | Reactive | tested | Reactive | Reactive |
| 50 | 48 | 48 | 100 | 12 | 12 | 100 |
| 16 | 48 | 44 | 91.7 | 12 | 11 | 91.7 |
| 5 | 48 | 18 | 37.5 | 12 | 4 | 33.3 |
| 1.6 | 48 | 7 | 14.6 | 12 | 2 | 16.6 |
| 0.5 | 48 | 0 | 0 | 12 | 0 | 0 |

Sensitivity of ZIKV PCR in plasma and urine on the cobas omni Utility channel on the cobas 6800 System

TABLE 5

| Sample | Ct-value | Ct-value |
|---|---|---|
| number | ZIKV PCR | Cobas Zika |
| 1 | 36.7 | 36.3 |
| 2 | 29.4 | 28.9 |
| 3 | 36.6 | 35.5 |
| 4 | 44.1 | 39.0 |
| 5 | 43.5 | 37.8 |
| 6 | 30.5 | 30.3 |
| 7 | 31.5 | 30.3 |
| 8 | 36.9 | 38.6 |
| 9 | 35.9 | 34.4 |
| 10 | 32.5 | 32.8 |
| 11 | 36.1 | 38.6 |
| 12 | 34.9 | 33.7 |
| 13 | 36.3 | 34.9 |
| 14 | NR | 38.0 |
| 15 | NR | 39.3 |
| 16 | NR | 38.3 |
| 17 | 39.2 | NR |
| 18 | 37.4 | NR |
| 19 to 29 | NR | NR |
| | 15/29 | 16/29 |

NR = non-reactive

1. A method of determining whether a sample comprises ZIKA virus (ZIKV) nucleic acid, the method comprising:
performing an amplification reaction with said sample in the presence of a second oligonucleotide set, a first oligonucleotide set, a third set or a combination of two or more of said sets, wherein:
the first oligonucleotide set has:
an oligonucleotide ON A with a consecutive stretch of at least 18 nucleotides of AARTACACATACCARAACAAAGTGGT (FP_ZIKA_San0-F) (SEQ ID NO:1) or a consecutive stretch of at least 18 nucleotides of TACACATACCARAACAAAGTGGT (FP_ZIKV_San0.alt-F) (SEQ ID NO:2); and
an oligonucleotide ON B with a consecutive stretch of at least 18 nucleotides of ACTTGTCCRCTCCCYCTYTGGTC (RP_ZIKA_San0-R) (SEQ ID NO:3);
the second oligonucleotide set has:
an oligonucleotide ON C with a consecutive stretch of at least 18 nucleotides of TGGTGTGGAAYAGRGTGTGGAT (FP_ZIKA_San-3F) (SEQ ID NO:4); and
an oligonucleotide ON D with a consecutive stretch of at least 18 nucleotides of GTAYTTYTCTTCATCACCTATG (RP_ZIKA_San-3R) (SEQ ID NO:5); and
the third oligonucleotide set has:
an oligonucleotide ON E with a consecutive stretch of at least 18 nucleotides of GTTGTGGATGGAATAGTGGT (FP_ZIKA_San-1F) (SEQ ID NO:6); and
an oligonucleotide ON F with a consecutive stretch of at least 18 nucleotides of AGTARCACYTGTCCCATCT (RP_ZIKA_San-1R) (SEQ ID NO:7),
wherein:
R=A or G; and
Y=C or T;
the method further comprising determining whether the sample comprises an amplification product of the amplification reaction.

2. The method of claim 1, wherein ON A comprises a consecutive stretch of at least 20 nucleotides of AARTACACATACCARAACAAAGTGGT (SEQ ID NO:1) or a consecutive stretch of at least 20 nucleotides of TACACATACCARAACAAAGTGGT (SEQ ID NO:2).

3. The method of claim 1, wherein ON B comprises a consecutive stretch of at least 20 nucleotides of ACTTGTCCRCTCCCYCTYTGGTC (SEQ ID NO:3).

4. The method of claim 1, wherein ON C comprises a consecutive stretch of at least 20 nucleotides of TGGTGTGGAAYAGRGTGTGGAT (SEQ ID NO:4).

5. The method of claim 1, wherein ON D comprises a consecutive stretch of at least 20 nucleotides of GTAYTTYTCTTCATCACCTATG (SEQ ID NO:5).

6. The method of claim 5, wherein ON E comprises a consecutive stretch of at least 20 nucleotides of GTTGTGGATGGAATAGTGGT (SEQ ID NO:6).

7. The method of claim 1, wherein ON F comprises a consecutive stretch of at least 19 nucleotides of AGTARCACYTGTCCCATCT (SEQ ID NO:7).

8. The method of claim 1, wherein:
ON A comprises AARTACACATACCARAACAAAGTGGT (SEQ ID NO:1) or the sequence TACACATACCARAACAAAGTGGT (SEQ ID NO:2);
ON B comprises ACTTGTCCRCTCCCYCTYTGGTC (SEQ ID NO:3);
ON C comprises TGGTGTGGAAYAGRGTGTGGAT (SEQ ID NO:4);
ON D comprises GTAYTTYTCTTCATCACCTATG (SEQ ID NO:5);
ON E comprises GTTGTGGATGGAATAGTGGT (SEQ ID NO:6); and/or
ON F comprises AGTARCACYTGTCCCATCT (SEQ ID NO:7).

9. The method of claim 1, wherein the determining whether the sample comprises an amplification product comprises incubating the amplified sample with one or more ZIKV specific probes that are specific for the amplification product of an amplification with the first oligonucleotide set, the second oligonucleotide set, the third oligonucleotide set or a combination of two or more of said sets.

10. The method of claim 9, wherein the one or more probes comprise:
an oligonucleotide with AAGGTYCTYAGACCA{G}{C}{T}{G}AA (SEQ ID NO:8);
an oligonucleotide with CGCA{C}CA{C}HTGGG{C}TGA (SEQ ID NO:9);
an oligonucleotide with ACTGACATTGACACAATGACAAT (SEQ ID NO:10);
an oligonucleotide with AAAT{G}{G}ACA{G}ACATTCCCTA (SEQ ID NO:11);
an oligonucleotide with the reverse complement of AAGGTYCTYAGACCA{G}{C}{T}{G}AA (SEQ ID NO:8);
an oligonucleotide with the reverse complement of CGCA{C}CA{C}HTGGG{C}TGA (SEQ ID NO:9);
an oligonucleotide with the reverse complement of ACTGACATTGACACAATGACAAT (SEQ ID NO:10);
an oligonucleotide with the reverse complement of AAAT{G}{G}ACA{G}ACATTCCCTA (SEQ ID NO:11); or
a combination of two or more of the oligonucleotides, wherein:
R=A or G;
Y=C or T; and
H=A or T or C, and
wherein the nucleotides indicated with parenthesis are preferably locked nucleic acid nucleotides and wherein the one more probes comprise two or more of the oligonucleotides, the two or more oligonucleotides preferably do not comprise oligonucleotides that are the reverse complement of each other.

11. The method of claim 9, wherein the step of determining whether the sample comprises an amplification product comprising monitoring amplification of the amplification product during the amplification process (real-time).

12. A kit comprising:
a second oligonucleotide set, a first oligonucleotide set, a third oligonucleotide set or a combination of two or more of said sets, wherein the first oligonucleotide set has:
an oligonucleotide ON A with a consecutive stretch of at least 18 nucleotides of AARTACACATACCARAACAAAGTGGT (FP_ZIKA_San0-F) (SEQ ID NO:1) or a consecutive stretch of at least 18 nucleotides of TACACATACCARAACAAAGTGGT (FP_ZIKV_San0.alt-F) (SEQ ID NO:2); and
an oligonucleotide ON B with a consecutive stretch of at least 18 nucleotides of ACTTGTCCRCTCCCYCTYTGGTC (RP_ZIKA_San0-R) (SEQ ID NO:3);
the second oligonucleotide set has:
an oligonucleotide ON C with a consecutive stretch of at least 18 nucleotides of TGGTGTGGAAYAGRGTGTGGAT (FP_ZIKA_San-3F) (SEQ ID NO:4); and
an oligonucleotide ON D with a consecutive stretch of at least 18 nucleotides of GTAYTTYTCTTCATCACCTATG (RP_ZIKA_San-3R) (SEQ ID NO:5); and
the third oligonucleotide set has:
an oligonucleotide ON E with a consecutive stretch of at least 18 nucleotides of GTTGTGGATGGAATAGTGGT (FP_ZIKA_San-1F) (SEQ ID NO:6); and
an oligonucleotide ON F with a consecutive stretch of at least 18 nucleotides of AGTARCACYTGTCCCATCT (RP_ZIKA_San-1R) (SEQ ID NO:7),
wherein:
R=A or G; and
Y=C or T.

13. A kit according to claim 12, further comprising one or more ZIKV specific probes that are specific for the amplification product of an amplification with the first oligonucleotide set, the second oligonucleotide set or a combination thereof.

14. An apparatus arranged for performing an amplification of Zika virus nucleic acid comprising a biological sample to be tested for the presence of Zika virus nucleic acid, and
one or more containers comprising:
an oligonucleotide ON C with a consecutive stretch of at least 18 nucleotides of TGGTGTGGAAYAGRGTGTGGAT (FP_ZIKA_San-3F) (SEQ ID NO:4); and
an oligonucleotide ON D with a consecutive stretch of at least 18 nucleotides of GTAYTTYTCTTCATCACCTATG (RP_ZIKA_San-3R) (SEQ ID NO:5);
one or more containers comprising:
an oligonucleotide ON A with a consecutive stretch of at least 18 nucleotides of AARTACACATACCARAACAAAGTGGT (FP_ZIKA_San0-F) (SEQ ID NO:1) or a consecutive stretch of at least 18 nucleotides of TACACATACCARAACAAAGTGGT (FP_ZIKV_San0.alt-F) (SEQ ID NO:2); and
an oligonucleotide ON B with a consecutive stretch of at least 18 nucleotides of ACTTGTCCRCTCCCYCTYTGGTC (RP_ZIKA_San0-R) (SEQ ID NO:3); and/or
one or more containers comprising:
an oligonucleotide ON E with a consecutive stretch of at least 18 nucleotides of GTTGTGGATGGAATAGTGGT (FP_ZIKA_San-1F) (SEQ ID NO:6); and
an oligonucleotide ON F with a consecutive stretch of at least 18 nucleotides of AGTARCACYTGTCCCATCT (RP_ZIKA_San-1R) (SEQ ID NO:7),
wherein:
R=A or G; and
Y=C or T.

15. The apparatus of claim 14 further comprising:
one or more containers comprising one or more probe oligonucleotides with AAGGTYCTYAGACCA{G}{C}{T}{G}AA (SEQ ID NO:8);
an oligonucleotide with the sequence CGCA{C}CA{C}HTGGG{C}TGA (SEQ ID NO:9);
an oligonucleotide with the sequence ACTGACATTGACACAATGACAAT (SEQ ID NO:10);
an oligonucleotide with the sequence AAAT{G}{G}ACA{G}ACATTCCCTA (SEQ ID NO:11);
an oligonucleotide with the reverse complement of the sequence AAGGTYCTYAGACCA{G}{C}{T}{G}AA (SEQ ID NO:8);
an oligonucleotide with the reverse complement of the sequence CGCA{C}CA{C}HTGGG{C}TGA (SEQ ID NO:9);
an oligonucleotide with the reverse complement of the sequence ACTGACATTGACACAATGACAAT (SEQ ID NO:10);
an oligonucleotide with the reverse complement of the sequence AAAT{G}{G}ACA{G}ACATTCCCTA (SEQ ID NO:11); or
a combination of two or more of the oligonucleotides, wherein:
R=A or G;
Y=C or T; and
H=A or T or C, and
wherein the nucleotides indicated with parenthesis are preferably locked nucleic acid nucleotides, and
wherein the one more probes comprise two or more of the oligonucleotides, the two or more oligonucleotides preferably do not comprise oligonucleotides that are the reverse complement of each other.
